(19) United States
(12) Patent Application Publication
(10) Pub. No.: US 2024/0389862 A1
(43) Pub. Date: Nov. 28, 2024

(54) MEASUREMENT DEVICE

(71) Applicant: Nippon Telegraph and Telephone Corporation, Tokyo (JP)

(72) Inventors: Yujiro Tanaka, Tokyo (JP); Daichi Matsunaga, Tokyo (JP)

(21) Appl. No.: 18/691,901

(22) PCT Filed: Oct. 21, 2021
(86) PCT No.: PCT/JP2021/038901

§ 371 Date: Mar. 14, 2024

(51) Int. Cl. A61B 5/01; A61B 5/00

(52) U.S. Cl. CPC A61B 5/01; A61B 5/7271; A61B 2560/0443; A61B 2560/0462; A61B 2562/0271

(57) ABSTRACT

The measurement device includes a measurement apparatus and a contact apparatus detachably attached to the measurement apparatus. The measurement apparatus includes a probe having one end disposed toward a target subject and made of a thermal resistor. The probe encloses a first temperature sensor and a second temperature sensor. The contact apparatus includes a thermally conductive structure, a first spacer, and a second spacer.

### CROSS-REFERENCE TO RELATED APPLICATIONS

This application is a national phase entry of PCT Application No. PCT/JP2021/038901, filed on Oct. 21, 2021, which application is hereby incorporated herein by reference.

### TECHNICAL FIELD

The present invention relates to a measurement device that measures a core body temperature.

### BACKGROUND

Recent research on chronobiology has revealed that circadian rhythms, so-called biological clocks, possessed by humans are closely related to various states of the human body, such as the quality of sleep, exercise and work, as well as the effect of medication and the onset of disease. The circadian rhythm is almost constant, but it is known that it changes greatly depending on the light people are exposed to in their daily lives, exercise, eating habits, age and gender.

Core body temperature is known as an index for measuring circadian rhythm. However, the core body temperature is generally measured by inserting a thermometer into the rectum or by measuring the temperature of the eardrum with the ear sealed. Accordingly, it is very stressful to measure the core body temperature in daily life or during sleep.

To solve this problem, a technology for estimating a core body temperature by a heat flux sensor using two sensors has been proposed (Non Patent Literature 1). This technology assumes a one-dimensional thermal equivalent circuit as illustrated in FIG. 10, and estimates the core body temperature from the heat flowing from the core of the body to the skin surface. From a skin temperature Tₛₖᵢₙ measured by the sensor and a temperature Tₜₒₚ of an upper portion of the sensor including a thermal resistor, the core body temperature T_{cbt}=Tₛₖᵢₙ+α(Tₛₖᵢₙ−Tₜₒₚ). Additionally, T_{cbt}=Tₛₖᵢₙ+αH_{Body} is established from a heat flux H_{Body} on the skin surface. A proportionality factor a can be obtained from the measurement results of other measurement devices for measuring the eardrum temperature, the rectum temperature and the like.

However, this technology has a problem that the core body temperature cannot be estimated because the heat does not flow linearly but flows to the surroundings due to fluctuating outside air temperature or wind in the surroundings. The estimation of the core body temperature by this technology is limited to use in a limited environment in a hospital, and is difficult to apply to a core body temperature monitor in daily life.

To solve this problem, another measurement device is proposed in which a heat flux from a target subject outside a sensor for measuring a skin temperature is transported to an upper portion of a probe where the sensor is built in, such that a one-dimensional heat flow is obtained even if there is a surrounding environmental fluctuation, and a change in thermal resistance between the sensor and outside air is suppressed (Non Patent Literature 2).

### Citation List

### Non Patent Literature

Non Patent Literature 1: H.—C. Gunga et al., “The Double Sensor-A non-invasive device to continuously monitor coretemperature in humans on earth and in space”, Respiratory Physiology & Neurobiology, 169S, pp. S63-S68, 2009.

Non Patent Literature 2: Y. Tanaka et al., “Robust Skin Attachable Sensor for Core Body Temperature Monitoring”, IEEE Sensors Journal, vol. 21, no. 14, pp. 16118-16123, 2021.

### SUMMARY

### Technical Problem

However, in the technology described above, since the sensor is in contact with the skin, it is not preferable to reuse the sensor for an unspecified number of people in terms of hygiene and risks of infection; it is desirable to use up the sensor. However, this type of measurement device includes an arithmetic circuit and a storage device necessary for communication with an external computer device and for estimating the core body temperature, and thus using up of the sensor has problems in terms of cost and the environment.

Embodiments of the present invention has been made to solve the problems described above, and an object of embodiments of the present invention is to measure a core body temperature in a more hygienic manner without incurring unnecessary cost and environment problems.

### Solution to Problem

A measurement device according to embodiments of the present invention includes: a measurement unit provided with a probe configured by a thermal resistor with a built-in sensor for obtaining temperature information of a target subject for which a core body temperature is to be measured, one end of which faces the target subject; and a contact unit detachably attached to the measurement unit, wherein the contact unit includes: a thermally conductive structure made of a thermally conductive material in the form of a cone-shaped cylinder; a first spacer made of a heat-insulating material and formed inside the thermally conductive structure and provided with a through-hole into which the probe is inserted and pulled out; and a second spacer made of a heat-insulating material formed to cover the outside of the thermally conductive structure, and the thermally conductive structure has a bottom surface side with a large area disposed on a side of the target subject, a top surface side disposed in contact with an other end side of the probe inserted into the through-hole, and transports heat flux from the target subject outside the probe to the other end of the probe.

### Advantageous Effects of Invention

As described above, according to embodiments of the present invention, since the contact unit attached to the measurement unit is detachable, the core body temperature can be measured in more hygienic manner without causing unnecessary cost and environment problems.

### BRIEF DESCRIPTION OF THE DRAWINGS

FIG. 1A is a configuration diagram illustrating a configuration of a measurement device according to an embodiment of the present invention.

FIG. 1B is a configuration diagram illustrating a configuration of the measurement device according to the embodiment of the present invention.

FIG. 2 is a configuration diagram illustrating a configuration of another measurement device according to an embodiment of the present invention.

FIG. 3 is a configuration diagram illustrating a partial configuration of another measurement device according to the embodiment of the present invention.

FIG. 4 is a configuration diagram illustrating a configuration of another measurement device according to an embodiment of the present invention.

FIG. 5 is a configuration diagram illustrating a partial configuration of another measurement device according to the embodiment of the present invention.

FIG. 6A is a configuration diagram illustrating a configuration of another measurement device according to an embodiment of the present invention.

FIG. 6B is a configuration diagram illustrating a configuration of another measurement device according to the embodiment of the present invention.

FIG. 7 is a configuration diagram illustrating a configuration of another measurement device according to an embodiment of the present invention.

FIG. 8 is a characteristic diagram illustrating a comparison result between a core body temperature (horizontal axis) measured and estimated by the measurement device according to the embodiment and a core body temperature (eardrum temperature: vertical axis) measured by an eardrum thermometer.

FIG. 9 is a characteristic diagram illustrating comparison of time-series changes between a core body temperature (solid line) measured and estimated by the measurement device according to the embodiment and a core body temperature (eardrum temperature: broken line) measured by the eardrum thermometer.

FIG. 10 is a circuit diagram illustrating a model of biological heat transfer by a one- dimensional thermal equivalent circuit.

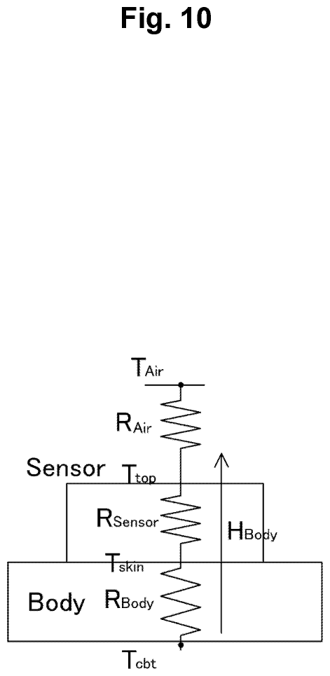

### DETAILED DESCRIPTION OF ILLUSTRATIVE EMBODIMENTS

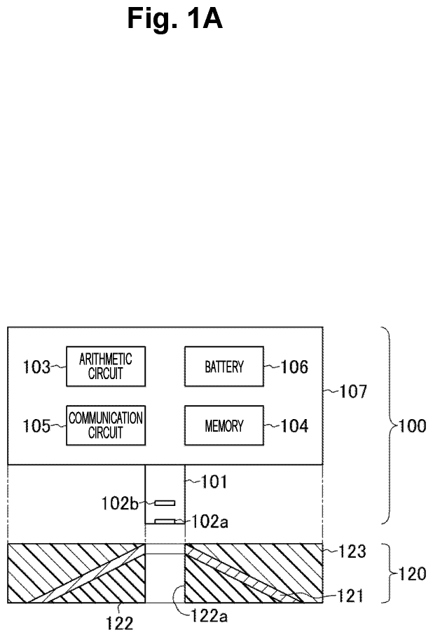

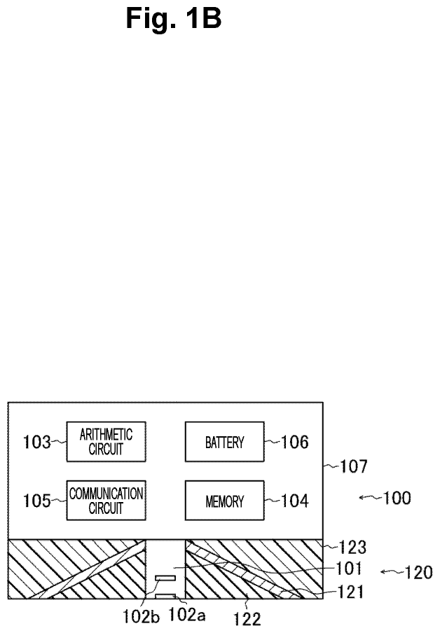

Hereinafter, a measurement device according to an embodiment of the present invention will be described with reference to FIGS. 1A and 1B. The measurement device includes a measurement unit 100 and a contact unit 120 detachably attached to the measurement unit 100. FIG. 1A illustrates a case where the contact unit 120 is combined with the measurement unit 100 to be in a measurement state, and FIG. 1B illustrates a state where the contact unit 120 is separated from the measurement unit 100. The contact unit 120 schematically shows a cross section in FIGS. 1A and 1B.

The measurement unit 100 includes a probe 101 having one end disposed toward a target subject and made of a thermal resistor. For example, in the measurement, a one end of the probe 101 comes into contact with the skin to be measured. On the paper of FIGS. 1A and 1B, a lower end of the probe 101 is referred to as the one end. The probe 101 incorporates a sensor for obtaining temperature information of the target subject for measuring a core body temperature. In this example, a first temperature sensor 102a and a second temperature sensor 102b are built in the probe 101 as sensors. The first temperature sensor 102a is disposed at the one end of the probe 101. The second temperature sensor 102b is disposed away from the first temperature sensor 102a in a direction away from the one end side of the probe 101.

The contact unit 120 includes a thermally conductive structure 121, a first spacer 122, and a second spacer 123. The thermally conductive structure 121 is made of a thermally conductive material (highly thermally conductive material) having a conical shape. In the thermally conductive structure 121, a bottom surface side having a large opening area is disposed on a side of the target subject, and a top surface side having a small opening area is disposed in contact with the other end side of the probe 101 inserted into a through-hole 122a, and a heat flux from the target subject outside the probe 101 is transported to the other end of the probe 101. On the paper of FIGS. 1A and 1B, an upper end of the probe 101 is referred to the other end. The probe 101 has, for example, a cylindrical outer shape, and the through-hole 122a has, for example, a cylindrical shape.

The thermally conductive structure 121 can be made of, for example, a metal such as aluminum. Further, the thermally conductive structure 121 can be constituted by, for example, a film material in which a thin metal film is laminated in multiple layers in a polymer, or a film material in which molecules having a very high thermal conductivity in a molecular bonding direction such as graphite are oriented at a high proportion in a polymer.

The first spacer 122 includes the through-hole 122a into and from which the probe 101 can be inserted and removed, and is formed inside the thermally conductive structure 121. The first spacer 122 is made of a heat-insulating material. The second spacer 123 is formed to cover the outside of the thermally conductive structure 121. The second spacer 123 is made of a heat-insulating material. The through-hole 122a can be tapered in a cross-sectional view such that an opening diameter gradually increases from an upper surface to a lower surface, thereby the inserted probe 101 being able to be held with an appropriate force.

Moreover, the measurement unit 100 includes, for example, an arithmetic circuit 103, a memory 104, a communication circuit 105 that functions as an I/F circuit with the outside, and a battery 106 that supplies power to the arithmetic circuit 103, and the communication circuit 105. Additionally, the measurement unit 100 includes a housing 107 incorporating the arithmetic circuit 103, the memory 104, the communication circuit 105 and the battery 106. The other end side of the probe 101 is fixed to an outer bottom surface of the housing 107. The second spacer 123 is disposed in contact with the outer bottom surface of the housing 107 around the other end side of the probe 101 inserted into the through-hole 122a.

The arithmetic circuit 103 estimates the core body temperature of the target subject from a measured value obtained by the first temperature sensor 102a and the second temperature sensor 102b using a predetermined equation. The memory 104 stores, for example, information on a one-dimensional biological heat transfer model based on the equation described above and an estimation result of the core body temperature. The memory 104 can be configured by a rewritable non-volatile storage device (for example, a flash memory). The arithmetic circuit 103 cooperates with a computer device such as an external smartphone connected via the communication circuit 105, estimates the core body temperature, and notifies a set notification destination of the estimated core body temperature.

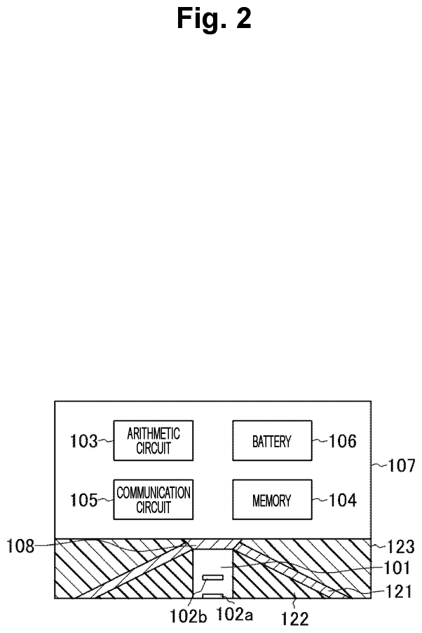

As illustrated in FIG. 2, the measurement device can include a plate-like thermally conductive portion 108 made of a thermally conductive material. In this case, the other end side of the probe 101 is fixed to the outer bottom surface of the housing 107 via the thermally conductive portion 108. A top surface side of the thermally conductive structure 121 is disposed in contact with the thermally conductive portion 108 on the other end side of the probe 101 inserted into the through-hole 122a. The contact unit 120 schematically shows a cross section in FIG. 2.

By using this measurement device, the core body temperature can be estimated as follows. First, a temperature measured by the first temperature sensor 102a is defined as a skin temperature Tₛₖᵢₙ of the target subject. A temperature measured by the second temperature sensor 102b is defined as a top temperature Tₜₒₚ of the probe 101 made of the thermal resistor. From these measurement results, the core body temperature can be estimated by the equation “core body temperature=Tₛₖᵢₙ+α(Tₛₖᵢₙ−Tₜₒₚ)” using a proportionality factor a (see Non Patent Literature 2). The proportionality factor a can be obtained from the measurement results of other measurement devices for measuring the eardrum temperature, the rectum temperature and the like. The calculation above is performed by the arithmetic circuit 103.

In this measurement device, since the contact unit 120 is detachably attached to the measurement unit 100, the contact unit 120 can be easily replaced with a new contact unit 120 after the core body temperature is measured once. As described above, according to the embodiment, since the contact unit 120 in contact with the skin in the measurement is used up, it is not necessary to replace the arithmetic circuit and the storage device necessary for estimating the core body temperature. Accordingly, according to the embodiment, the core body temperature can be measured in more hygienic manner without causing unnecessary cost and environment problems.

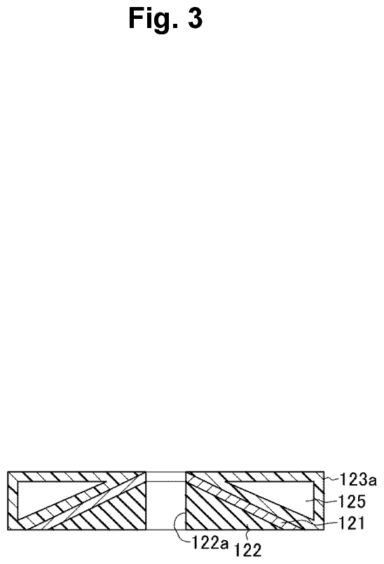

As illustrated in FIG. 3, the second spacer 123 may have a hollow structure including a hollow portion 125. The second spacer 123 can include the hollow portion 125 within a range in which an appropriate force for holding the probe 101 inserted into the through-hole 122a can be obtained. The arrangement of the hollow portion 125 is desirably rotationally symmetric in plan view such that heat transport is one-dimensional.

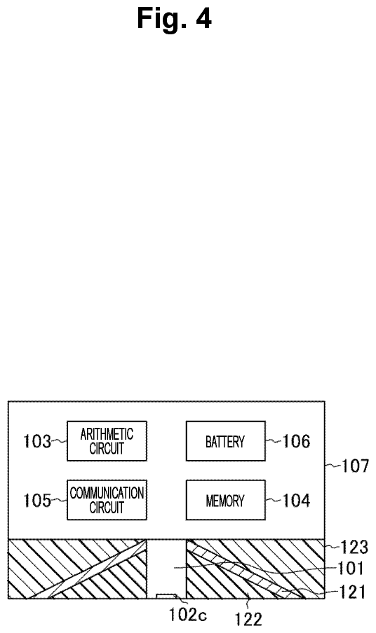

As illustrated in FIG. 4, a sensor 102c including a temperature sensor and a heat flux sensor can be disposed (incorporated) at the one end of the probe 101 as a sensor for obtaining temperature information of the target subject for measuring a core body temperature. The contact unit 120 schematically shows a cross section in FIG. 4.

By using this measurement device, the core body temperature can be estimated as follows. First, a temperature measured by the temperature sensor 102c is defined as a skin temperature Tₛₖᵢₙ of the target subject. A heat flux measured by the sensor 102c is defined as a heat flux H_{Body} on the skin surface of the target subject. From these measurement results, the core body temperature can be estimated by the equation “core body temperature=Tₛₖᵢₙ+αH_{Body}” using a proportionality factor ⊕ (see Non Patent Literature 2). The proportionality factor ⊕ can be obtained from the measurement results of other measurement devices for measuring the eardrum temperature, the rectum temperature and the like. The calculation above is performed by the arithmetic circuit 103.

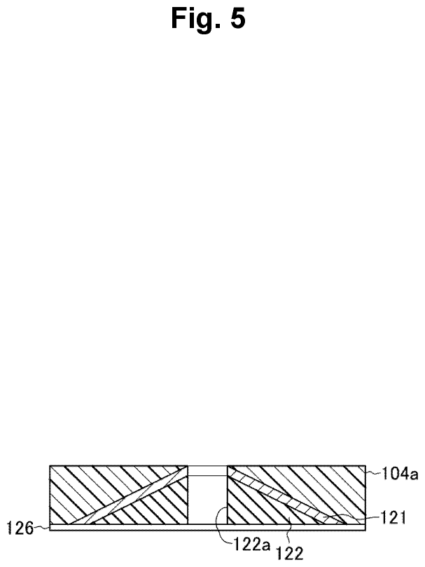

As illustrated in FIG. 5, a heat transfer sheet 126 can be formed on a contact surface of the first spacer 122 in contact with the target subject. The heat transfer sheet 126 can be formed to cover (enclose) the through-hole 122a in the entire region of a surface of the first spacer 122 on a side in contact with the target subject. The heat transfer sheet 126 can be made of, for example, the same material as the thermally conductive structure 121. By providing the thermally conductive structure 121, the probe 101 and the living body do not come into direct contact with each other, which is more desirable in terms of hygiene.

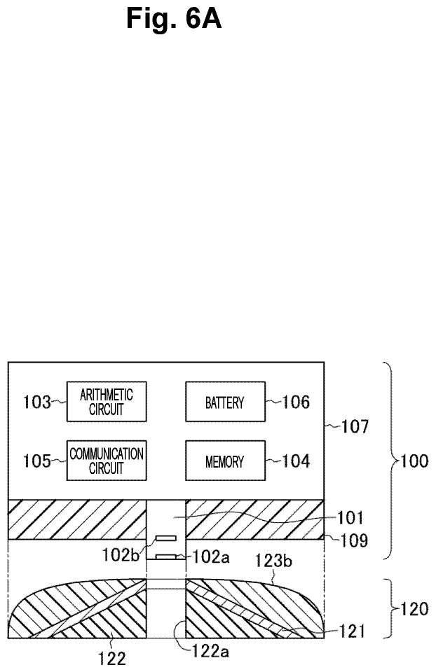

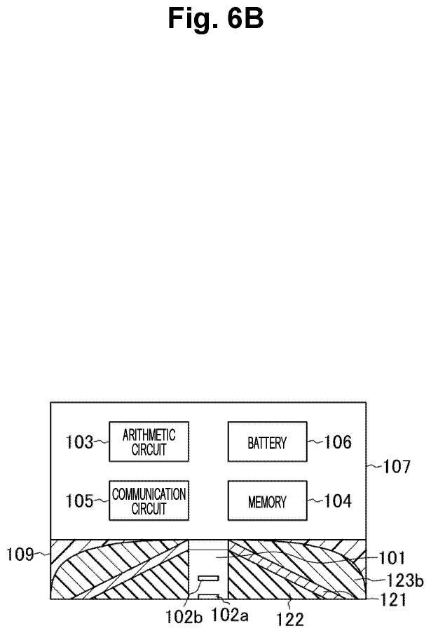

As illustrated in FIGS. 6A and 6B, the contact unit 120 may further include a buffer member 109 formed on the outer bottom surface of the housing 107 around the probe 101. In this case, it is possible to use a second spacer 123a in which a surface facing a side of the measurement unit 100 is a three-dimensional convex curved surface such as a part of a spherical surface. FIG. 6A illustrates a case where the contact unit 120 is combined with the measurement unit 100 to be in a measurement state, and FIG. 6B illustrates a state where the contact unit 120 is separated from the measurement unit 100.

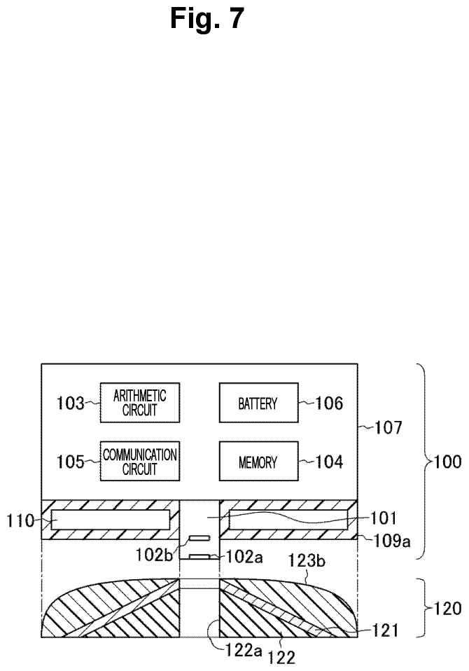

The buffer member 109 can be made of, for example, a polymer elastic fiber. In a case where the contact unit 120 is made of a flexible material, the contact unit 120 is deformed in accordance with a complicated aspect of the living body of the target subject. In such a case, the buffer member 109 is deformed in accordance with the deformation of the contact unit 120. As illustrated in FIG. 7, a buffer member 109a having a hollow structure including a hollow portion 110 can also be used. In FIGS. 6A, 6B, and 7, the contact unit 120, the buffer member 109, and the buffer member 109a schematically show cross sections.

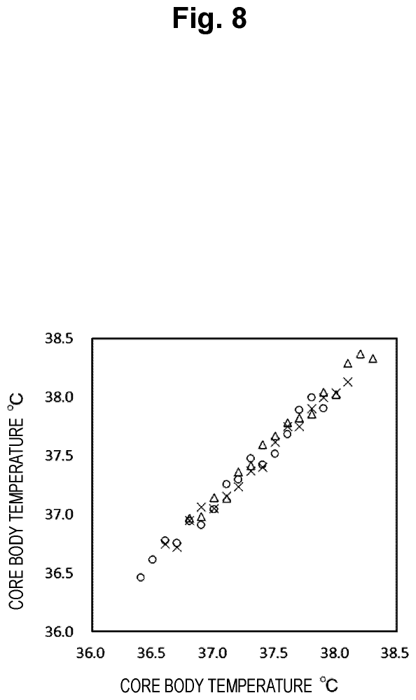

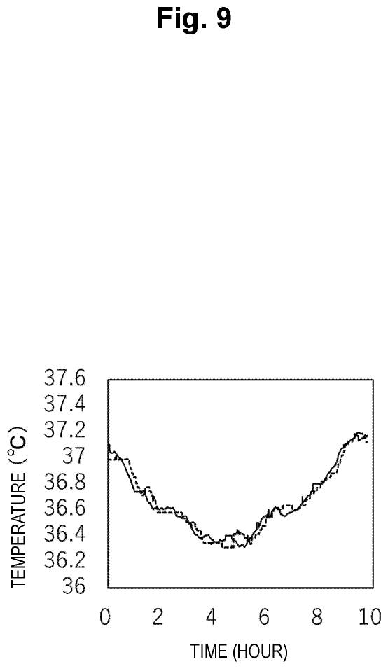

FIG. 8 illustrates a comparison result between a core body temperature (horizontal axis) measured and estimated by the measurement device according to the embodiment and a core body temperature (eardrum temperature: vertical axis) measured by an eardrum thermometer. FIG. 9 illustrates comparison of time-series changes between a core body temperature (solid line) measured and estimated by the measurement device according to the embodiment and a core body temperature (eardrum temperature: broken line) measured by the eardrum thermometer.

As illustrated in FIGS. 8 and 9, it can be seen that a result close to the result measured by the eardrum thermometer is obtained by the measurement device according to the embodiment.

As described above, according to embodiments of the present invention, since the contact unit attached to the measurement unit is detachable, the core body temperature can be measured in more hygienic manner without causing unnecessary cost and environment problems.

Note that the present invention is not limited to the embodiments described above, and it is obvious that many modifications and combinations can be implemented by a person skilled in the art without departing from the technical idea of the present invention.

### REFERENCE SIGNS LIST

100 Measurement unit
101 Probe
102a First temperature sensor
102b Second temperature sensor
103 Arithmetic circuit
104 Memory
105 Communication circuit
106 Battery
107 Housing
120 Contact unit
121 Thermally conductive structure
122 First spacer
122a Through-hole
123 Second spacer

1-8. (canceled)

9. A measurement device, comprising:
a measurement apparatus provided with a probe configured by a thermal resistor with a built-in sensor for obtaining temperature information of a target subject for which a core body temperature is to be measured, one end of which faces the target subject; and
a contact apparatus detachably attached to the measurement apparatus,
wherein the contact apparatus includes:
a thermally conductive structure made of a thermally conductive material, the thermally conductive material being a cone-shaped cylinder;
a first spacer made of a heat-insulating material and formed inside the thermally conductive structure and provided with a through-hole into which the probe can be inserted and pulled out; and
a second spacer made of a heat-insulating material formed to cover the outside of the thermally conductive structure, and
the thermally conductive structure has a bottom surface side with a large area disposed on a side of the target subject, a top surface side disposed in contact with an other end side of the probe inserted into the through-hole, and transports heat flux from the target subject outside the probe to the other end of the probe.

10. The measurement device according to claim 9, wherein the measurement apparatus includes:
an arithmetic circuit that estimates the core body temperature of the target subject from a measured value obtained by the sensor; and
a housing accommodating the arithmetic circuit, and the other end side of the probe is fixed to an outer bottom surface of the housing.

11. The measurement device according to claim 10, wherein
the other end of the probe is fixed to the outer bottom surface of the housing via a plate-shaped thermally-conductive portion made of a thermally conductive material, and
the top surface side of the thermally conductive structure is arranged in contact with the thermally conductive portion on the other end side of the probe inserted into the through-hole.

12. The measurement device according to claim 10, wherein the second spacer is disposed in contact with the outer bottom surface of the housing around the other end side of the probe inserted into the through-hole.

13. The measurement device according to claim 12, wherein the contact further includes a buffer member formed on the outer bottom surface of the housing around the probe.

14. The measurement device according to claim 13, wherein the buffer member has a hollow structure.

15. The measurement device according to claim 9, wherein the second spacer has a hollow structure.

16. The measurement device according to claim 9, further comprising:
a heat transfer sheet formed on a contact surface of the first spacer in contact with the target subject.

17. A measurement device, comprising:
a measurement apparatus including a probe having a sensor including a temperature sensor and a heat flux sensor disposed at one end of the probe; and
a contact apparatus detachably attached to the measurement apparatus, the contact apparatus including a thermally conductive structure, a first spacer, and a second spacer, the thermally conductive structure made of a thermally conductive material and having a bottom surface side with a large area disposed on a side of a target subject, a top surface side disposed in contact with an other end side of the probe inserted into a through-hole of the first spacer, and configured to transport heat flux from the target subject outside the probe to the other end of the probe.

18. The measurement device of claim 17, wherein the thermally conductive structure is made of a metal.

19. The measurement device of claim 18, wherein the metal is aluminum.

20. The measurement device of claim 17, wherein the first spacer and the second spacer are made of a heat-insulating material.

21. The measurement device of claim 17, further comprising:
an arithmetic circuit configured to estimate the core body temperature of the target subject from a measured value obtained by the temperature sensor and the heat flux sensor using a predetermined equation.

22. The measurement device of claim 21, further comprising:
a memory configured to store information on a one-dimensional biological heat transfer model based on the predetermined equation and an estimation result of the core body temperature.
